(19) United States
(12) Patent Application Publication
(10) Pub. No.: US 2010/0179623 A1
(43) Pub. Date: Jul. 15, 2010

(54) HAND-HELD DEVICE AND METHOD FOR THERMAL TREATMENT OF AN AREA AFFECTED BY AN INSECT STING OR BITE

(75) Inventors: Norbert Hofer, Rothenbach (DE); Peter Heindl, Pyrbaum (DE)

Correspondence Address: RANKIN, HILL & CLARK LLP, 38210 Glenn Avenue, WILLOUGHBY, OH, 44094-7808, US

(73) Assignee: E.I.S. GMBH, Schwaig (DE)

(21) Appl. No.: 12/160,723

(22) PCT Filed: Jan. 8, 2007
(86) PCT No.: PCT/EP07/00092

§ 371 Date: Jul. 11, 2008

(30) Foreign Application Priority Data
Jan. 13, 2006 (DE) 102006001889.3
Apr. 27, 2006 (DE) 102006019552.3

(51) Int. Cl. A61F 7/08

(52) U.S. Cl. 607/96

(57) ABSTRACT

Different designs of hand-held devices for therapeutic skin treatment by application of heat are known. Inexpensive hand-held devices use heat radiation. In practice, however, there is no inexpensive hand-held device available that can be placed on the skin to be treated and that operates according to the principle of heat conduction and allows the skin to be treated within a defined temperature range without the use of a temperature sensor and control elements. To provide the user with an easy-to-use hand-held device that is inexpensive to produce and is based on the principle of heat conduction, the hand-held device according to the invention for thermal treatment of an area affected by an insect sting or bite comprises: a heat-conducting body (K) which is mounted in the housing (G) of the hand-held device and can be brought into contact with the skin (H) of the user, a temperature-dependent resistor which is arranged on or in the heat-conducting body (K) and is thermally coupled to the latter and has a positive characteristic curve (PTC), a keypad element (T) that can be manually actuated by the user, and a voltage source (B) arranged in the housing (G) of the hand-held device, such that, depending on the duration of actuation of the keypad element (T) and depending on the heat transfer resistances (R_{TH1}, R_{TH2}, R_{TH3}) between temperature-dependent resistor (PTC), heat-conducting body (K) and skin (ID the heat-conducting body (K) on the skin contact surface (HK) can be heated by the heat of the temperature-dependent resistor (PTC) to a temperature in the temperature range between 50° C. and 65° C. The invention applies to the field of medical equipment.

The invention concerns first a hand-held device for thermal treatment of areas affected by insect stings or bites according to patent claim 1. In addition the invention concerns also a method to treat areas affected by insect stings according to patent claim 10.

Stings or bites by insects like mosquitoes, louses, hornets, bugs or ants cause most frequently itching, sometimes painful but quickly fading in most cases tumefactions. Such stings of bites are harmful first of all by driving infections, poisons, blood thinning substances, etc., in the body and are extremely unpleasant for most of the affected persons. Itching, tumefactions and skin irritations affect as a rule stung or bitten areas only but allergic reactions may spread throughout the whole body.

Known methods of prophylaxis against insect stings that, however, can not be prevented, are:
mechanical protective means like window gratings, meshes, close clothes;
devices and units generating noise or scents like the ones of the contrary, male insects “capturing” thus bloodsucking female insects;
means on basis of various fragrances keeping insects away.

Likewise, known methods for treatment of already existing stings or bites are:
soothing affected area by cooling and/or creams or alcohol with soothing and/or anti-allergic effect but with temporary only lulling of bite symptoms meaning external chemical treatment of affected areas;
thermal treatment and (at least partial) neutralization and removal thereby of thermo-labile poisons through thermal intervention with temperature up to 70° C. at maximum.

The invention framework covers the last above mentioned method. As example, a device is known from EP 1 231 875 utilizing thermal treatment of consequences of insect bites and stings. To produce a device driving heat volumes as required for neutralization of thermo-labile poisons independently of the ambient conditions and preventing skin irritation due to heat effect, such a device must incorporate a heating element made in the form of heating plate supplied from energy source whereat the heating plate can be heated tin a stage to maximum temperature within the borderlines from 50° C. up to 65° C., and best from 55 to 60° C. and such a maximum temperature should be maintained within a stage for a period from 2 sec up to 12 sec, preferred from 3 sec up to 6 sec. To this purpose the heating element is coupled with a temperature sensor and a control unit with electric signal emitted from the temperature sensor delivered to the control unit governing the plate heating to maximum temperature and duration of such maximum temperature. The heating element in one of the design versions and the temperature sensor are integrated in a diagram of a silicon chip with actuating transistors whereat the heating element is made as a resistive matrix. Actuation of a button by the control unit heating element initiates the stage of plate heating. The heating stage starts when an electric signal emitted by the sensor reaches a preset maximum temperature for a heating plate of relevant size. Therefore, a time control is actuated in the control unit containing a temperature and time governor. The temperature governor ensures maintenance of maximum temperature and simultaneously prevents rising thereof during a preset heating stage corresponding to the process duration. The heating process ends with expiration of the heating stage and heating plate temperature is adapted to the ambient temperature. Example: a heating plate is heated during the heating stage to temperature in the range from 50° C. up to 65° C., preferred 55° C. to 60° C., observing simultaneously the deviation limits of ±3° C. During the stage following the heating stage equal to the process duration the maximum temperature is maintained for a period from 2 up to 12 seconds, preferred 3 to 6 seconds. The heating stage is inversely proportional to the maximum temperature. The heating process ends with the heating stage termination and the cooling stage begins. The heating stage end and thereby the end of the process itself may be registered by an optic signal. Driving heat energy in the bite area is made therefore independently of the ambient temperature and the skin surface temperature since the heating stage starts only after the maximum temperature is reached while the heating plate temperature is maintained constant throughout the heating stage by means of the temperature governor. Thus is possible to deliver heat energy within preset temperature limits needed to neutralize the thermo-labile poisons without damaging the skin by such thermal energy.

A similar process is applied in the hand-held device known from DE 101 34 878 A1 for thermal treatment of skin areas affected by insect stings. The hand-held device may as example be made under the form of a thin cylindrical pocket light with heating head which, supplied with electricity from a battery or from the mains, can be pressed heated onto the affected skin area (the sting spot). The temperature of hand-held device section pressed onto the respective skin area can be adjusted as the skin will not burn but will reach thermal decomposition of injected poison (poison effect reduction). The hand-held device operation method compliant with the invention consists therefore in warming the heating head up activating thereof and then, preferably through strong pressing, applying it onto the sting affected area.

Besides, EP 0 312 413 A1 presents another hand-held device for therapeutic skin treatment through heat driving in where heat is generated by a heating spiral with capacity of 1.25 W placed over the skin surface (as example, at approximately 2 mm distance). Direct contact between skin and heating spiral is prevented by a wire screen (mesh size 0.3 mm) installed in front of orifice with 6 mm diameter and heat radiation can be increased with the help of a reflector mounted behind the heating spiral. The heating spiral temperature is not adjustable and electric supply is provided by a 9V battery. The spiral is warmed up by pressing a button and a LED indicates optically pressed position of the button.

A similar process is applied in the hand-held device, known from JP 02104351 A, for thermal treatment of sting or bite affected skin areas. The hand-held device is designed as a spherical support surface with a manually actuated switch mounted on the opposite side. A heating filament is intended for heating element, especially a NiCr-wire placed inside the round ring of the device base and electric supply is provided by a battery mounted inside the device. Thermal treatment is achieved through thermal irradiation (no direct contact with skin) from the filament and the temperature is impossible for governing.

EP 0 607 472 A1 introduces a device in the form of a disc for thermal treatment of skin areas affected by insect bites without own electric supply but charged by a charger. The heating process starts at the moment when the inside accumulating capacitor is charged up and ends when the same capacitor discharges completely. The charging process may be assisted by a microcomputer mounted in the device (with one supply battery) for control of the charging process switching the charger on and off and of the temperature. Alternatively the device control may be aided also by a manually activated switch combined with a thermostat or by a time relay combined with a switch. The heating process starts by pressing the switch whereat an ohmic heater is warming controlled a plate and thermal treatment is realized upon contact/placing the disc device onto the affected skin area.

FR 2 746 296 A1 introduces a square manual device for therapeutic treatment through heat injection. Heat is accumulated from power losses of a powerful transistor (as example, TO 220) built in the device and is applied onto the skin through a ceramic or thin metal (copper, aluminum) plate disposed in the one corner of the device beveled at 45° angle. Switching on is effected through touch-sensitive contacts installed also in the beveled corner. Temperature is measured by external temperature sensor or on the powerful heating transistor itself. Transistor power losses and as result thereof the plate temperature can be increased controlled in stages by actuation of auxiliary installed components. The device performance is registered by optic equipment.

A similar method is applied in the cylindrical device, known from JP 1022995 A, for treatment of skin rashes. Actually, the device consists of a battery, a temperature governor, a heating element and an aluminum plate with silver coating. The heating element is warmed to temperate within set limits: the skin rach is treated through contact with the affected skin area and heating. Adhesive layer is applied on the heating element surface for longer procedures. An alternative version provided for a distant element to be mounted between the skin surface and the heating surface and treatment of the skin surface by steam.

Last, US 2004/0127862 A1 introduces also a cylindrical hand-held device for treatment of skin rashes and skin diseases by controlled delivery of heat to skin. Common of the different versions are electric supply, temperature sensor, heating assembly, combined governing of temperature and time and various service elements setting desired temperature and time values. The heating assembly in one of the versions includes either a heating plate of conductive material (as example, metal) to be placed on the skin or a heating metal plate coated on the service side by plastic. The heating assembly in another version consists of heat-conductive fluid conveying heat toward the heating plate with heating head of plastics or rubber with rounded contact surface. In another version the skin surface to be treated is sprayed by warm fluid and the hand-held device itself disposes of several chambers with different solutions or mixture thereof respectively. If sprayed fluid contains alcohol, the skin is warmed up quickly and is cooled while the alcohol is evaporating. Hand-held devices with heating plates of larger diameters are connected to and supplied from the mains. For heat source may be used, as example, a laser, microwaves or infrared light. The desired temperature of the device is registered by optic equipment. Time and temperature control starts simultaneously with placing the device on the skin.

As this examination of engineering demonstrates, there are various equipped hand-held devices for therapeutic skin treatment through heat forcing in. Cheap devices like the ones of EP 0 312 413 A1 or EP 0 312 413 A1, operate with heat irradiation. But in practice there is not any cheap and placeable on skin device operating on principle of heat-conductivity and allowing without any temperature sensors, control and governing elements, to treat skin within preset temperature ranges. It is of particular significance since the industry manufacturing medical equipment is considered for a very advanced and fast developing industry adopting quickly any improvement and simplification and applying the same in practice.

This invention unlike the known hand-held devices is grounded on the task to update the same so that consumer is supplied with an easy to handle and cheap device allowing, on the principle of heat conductivity, to treat skin within preset temperature ranges at the spot of insect sting or bite.

The above task is settled by invention of a hand-held device for thermal treatment of skin affected by insect sting or bite, according to patent requirement 1, incorporating the following components
a heat-conductive body incorporated in the hand-held device housing entering in contact with the consumer's skin;
a temperature dependent resistor with positive curve disposed on or in the heat-conductive body and coupled thermally with the latter;
one button component actuated manually by the consumer, and
a voltage source installed in the hand-held device housing.

so that, depending on the duration of the button component pressing and on the thermal resistances between the temperature dependant resistor, the heat-conductive body and skin, the heat-conductive body of the skin contact surface be able to be warmed by the own heat of the temperature dependant resistor to temperature ranging from 50° C. to 65° C.

The hand-held device, subject of the invention, has the advantage to offer an unexpectedly easy and cheap method for heat treatment of insect bites and stings utilizing a temperature-dependant resistor in a single design element without any auxiliary control and governing elements incorporates both heating and governing with low expenditures on the basis only of its physical properties (positive σ−R-parameter). The very cheap realization is based on the governing features of the temperature-dependant resistor and on the elimination of additional sensor, especially of temperature sensor and governors as the engineering standard demands.

In addition, such task is solved in compliance with the invention after a method for thermal treatment of areas affected by insect stings and bites with the help of a hand-held device comprising of a temperature-dependant resistor with positive characteristic, a heat-conductive body coupled thermally with the resistor, a voltage source and a button component actuated manually by user according to patent requirement 10, whereat
user places the device onto the skin and
then presses the button component for thermal treatment of affected area as long as the self-heating temperature-dependant resistor gains acceptable temperature ranging within 50° C. and 65° C. at the contact between skin and heat-conductive body.

The method, subject to the invention, has the advantage to perform thermal treatment very simplified and fast while the user governs it individually by pressing on the button. Low power consumption is another advantage of the method since user presses the button as long as required for the manipulation itself.

For further invention updating, according to patent requirement 2, the body is made hollow with temperature-dependant resistor in the form of disc washer with two terminals. The disc washer is positioned in the hollow section of the heat-conductive body filled partially by heat-conductive material.

Such updating of the invention has the advantage to produce a compact heating element consisting of a hollow body and temperature-dependant resistance with low production cost with reduced expenditures for the hand-held device installation.

The temperature-dependant resistance in one preferred version of the invention, according to patent requirement 3, is fixed to a section of the housing by means of completely insulation applied through extrusion.

That version of the invention has the advantage to facilitate in unexpectedly easy manner safe fastening and good protection of the temperature-dependant resistance to the housing coating on the one hand and heat transfer to the body on the other hand. Such components are cheap to produce automatically and easy to mount in the hand-held device housing ensuring especially efficient transfer of heat.

For both the formation of the housing as a hollow body and disposition of resistor inside (patent requirement 2) and assembling the housing by inter-joined corresponding single components, as example plastic parts and mounting the resistor on the outside of the housing (patent requirement 3), heat distribution within the housing can be realized as the plastic part whereto the resistor is fastened or in contact with respectively, has at least one zone of increased thermal conductivity and the same is produced as a component of heat-conductive plastics through multi-component pressure casting by means of injection molding or pouring substitutes of heat-conductive plastics or metal. Additionally, to improve thermal conductivity, plastics and metal or ceramic powders could be combined. In cases where metal powder is used because increase of electric conductivity of plastics is not desired (due to its effect on the resistor terminals) could be used ceramic fillers like siliceous oxide, aluminum oxide or beryllium oxide. Purposefully inserted anisotropies of heat-conductivity with preferred direction, as example filament fillers, serve for guided conveyance of heat through the component on the housing. As an alternative it is possible to operate by one component of injection molding and plastics to be added with previously prepared substitutes of thermally conductive plastics or ceramics of metal, as example magnesium or aluminum.

Other advantages and details could be identified in the following description of preferred versions of invention basing on the drawings. On the drawings:

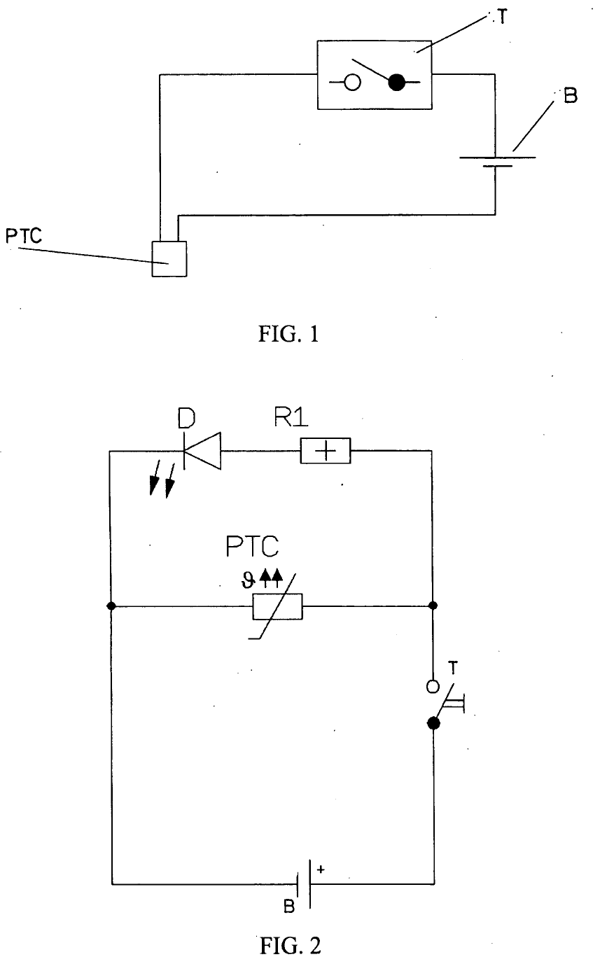

FIG. 1 shows block diagram

FIG. 2 shows electric diagram of the hand-held device

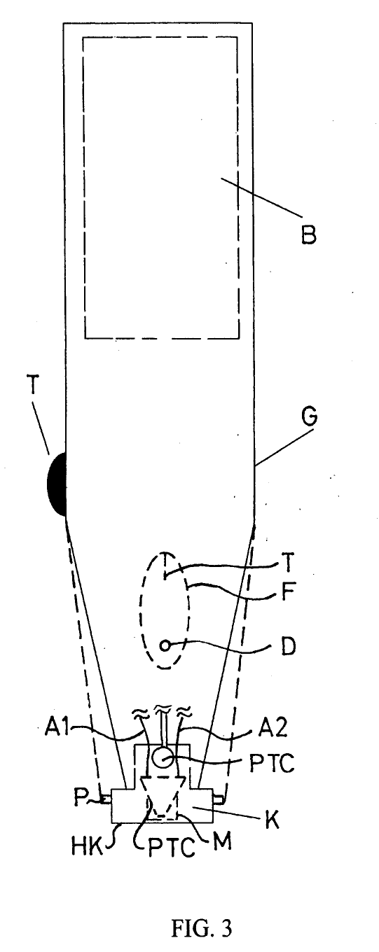

FIG. 3 shows various versions of the hand-held device viewed from the side

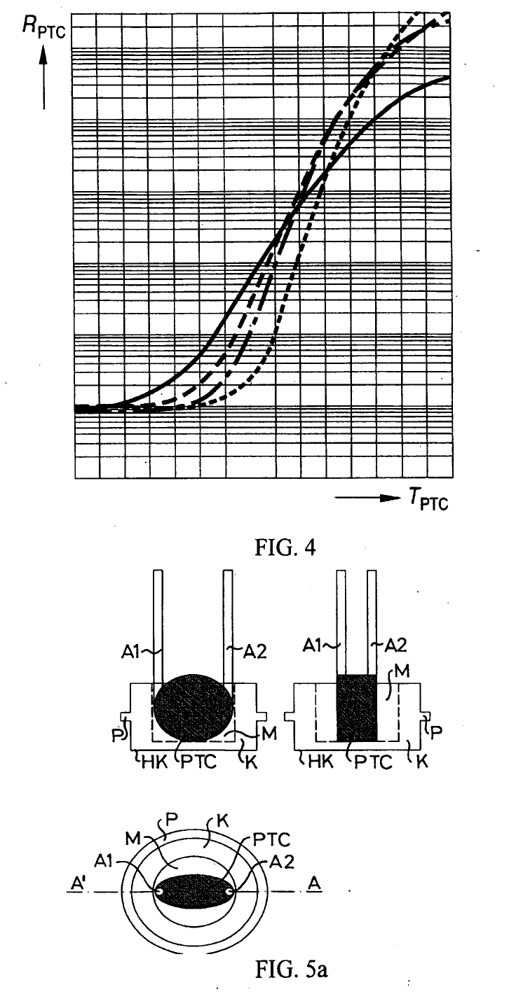

FIG. 4 shows resistance curve of PTC with different rated temperature

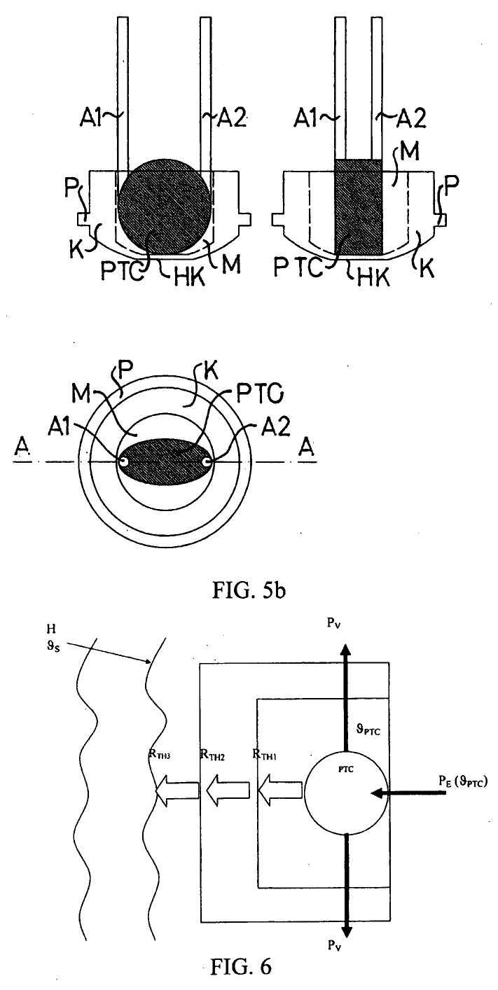

FIGS. 5a, 5b show two versions to install temperature-dependant resistor in the housing

FIG. 6 shows principle of heat transfer on the basis of heat-transfer resistors.

FIG. 1 through FIG. 6 are showing block diagram, electric diagram, resistance curve of PTC (positive temperature coefficient resistor) and the relative principle of heat transfer in the various versions of the hand-held device as subject of the invention for thermal treatment of areas affected by insect stings or bites. All these devices consist mainly of one temperature-dependant resistor with positive PTC characteristics, one coupled thereto thermal heat-conductive body (K), one energy source (B) and one button component actuated manually by user.

Heat is propagating by thermal irradiation, heat transfer and thermal stream (convection). Thermal radiation facilitates also heat transfer in vacuum depending on the temperature only of radiating body and not on the ambient temperature. Heat radiation depends on the radiator/housing and on the properties of the radiator surface. Heat conductivity is realized in matter only meaning in the body and required temperature drop in the body. Whenever a body on certain spot resists to or continuously abstracts heat, then temperature differences in the body equalize in the course of time and with a heat stream flowing from higher to lower temperatures. Metals are relatively good heat conductors: as example, copper has heat conductivity of 0.93 cal/cm-s-k within the temperature range from 0 up 100° C., aluminum has heat conductivity of 0.55 cal/cm-s-k within the temperature range from 0 up to 200° C., while plastics as a rule are poor conductors of heat. Heat transfer from a body with specified temperature toward the environment is described by the heat conductivity coefficient while heat transfer through a body, for example a plate, is described by the heat transfer coefficient. The heat conductivity coefficient depends strongly, as explained above, on the surface characteristics while the heat transfer coefficient depends on the material thickness (plate thickness). The hand-held device according to the invention has the advantage to optimize unexpectedly easy and cheap the ratio between the heat conductivity coefficient and the heat transfer coefficient to extent (through spraying of a part of the housing and the thermal capacity thereof, respectively the thermal capacity of the heat conductive material) ensuring especially efficient conveyance of heat.

The block diagram on FIG. 1 presents structural (design) components and FIG. 2 presents electric diagram of the hand-held device subject to the invention. According to the invention, the heat capacity of the temperature dependant resistor with positive characteristics PTC is driven by suitable contact into the user's skin H (see FIG. 6). Suitable contact could be made by mounting the PTC resistor into (see FIG. 3, FIG. 5a and FIG. 5b) or on the heat conductive body K (see FIG. 3), as example a cylindrical metal heating plate with diameter from 5 mm up to 7 mm. The temperature dependant resistor PTC is supplied from the voltage source B (battery, storage battery with voltage of 9V for fast warming of the PTC resistor). The temperature dependant resistor is switched with actuation o the button component T and the current flow through the PTC resistor is maintained until the manipulation is terminated by the button component release.

At first, due to lower cold resistance of the temperature dependant resistor PTC flows greater current causing subsequently self-warming of the resistor PTC. When the PTC resistor reaches a specified temperature value the PTC resistance raises fast and limits the current flow and thereby the loss capacity arising in the PTC resistor.

Further on, the temperature of the temperature-dependant resistor PTC specified only by typical properties of PTC and the temperature of the thermally coupled body K are raising on the basis of the governing property (see FIG. 4 presenting the resistively travel of PTC under various rated temperatures).

By optimization corresponding to the invention (dimensioning of heat transport especially between temperature-dependent resistor PTC and body K) is gained surface temperature of body K of about 50-65° C., preferable from 50-60° C., with temperature limits depending on the PTC and on the heat transfer. Dimensioning of pre-resistor R1 ensures protection against inadmissible temperatures harmful to the user, see FIG. 2.

FIG. 3 presents various versions of the hand-held device viewed from the side. Body K of the first version is made as a hollow body and the temperature-dependant resistor PTC is formed as blunt pyramid with two terminals A1 and A2 whereat the pyramid is positioned with least partial application of heat conductive material M in the hollow space of the heat conductive body K. The temperature-dependant resistor PTC in the second version has approximate shape of a disc washer (standard trade shape) and is mounted on the external side of the body K. In the first version thus formed component is entered from the front into the holder of the housing G of the hand-held device and is fastened while in the second version on the coating of body K there is profile P directed outwards and inwards with the profile of housing G mounted thereto (presented on FIG. 3 by dotted line). Housing G consists mainly of two plastic half-shells (not shown on the drawings) whereat, to facilitate mounting, the first half-shell of the housing is provided with partition wall between the battery B and the structural component of hand-held device. The second half-shell also is divided in two with the first section closing the structural components of the device joined by locking unit (and/or by adhering) to the first half-shell. The second section of the second half-shell rests in the first half-shell allowing movement and corresponding to the shape of the first section. The hand-held device is provided with LED or light D for optical indication when pressing the button. The above indicators are disposed behind a window F resting on a pin of the first semi-shell and held elastically by a spring. At the same time in the inside there is a shaped appliance (as example, a molding) actuating the button component T (shown on FIG. 3 by dotted line).

The temperature-dependant resistor PTC of the designs shown on FIG. 5a and FIG. 5b is made approximately in the form of disc washer with two terminals A1 and A2 and is in immediate contact with body K in the zone of the skin contact surface HK (FIG. 5a, elliptic section, FIG. 5b—round section). To reduce the heat transfer resistance and to optimize the thermal capacity the skin contact surface HK of body K in the first version presented on FIG. 5b is made convex with smaller thickness of the material. Basing on the structure of the temperature-dependant resistor PTC and aiming to simplify production of body K as a rotating component, preferable as a hollow cylinder opening from one side only, and to simplify installation, the body is adhered vertical in the hollow cylinder by means of heat conductive material M. The adhesive presents epoxy-based resin of good heat conductivity, insignificant compressibility and hardening at ambient temperature.

The disc-like shape of temperature-dependant resistor PTC ensures relatively higher thermal resistance compared to the skin contact surface HK whereby the heat energy generated in PTC is transferred toward the contact surface HK mainly.

The body K consists of good heat-conductive metal (copper or aluminum) that is gold plated due to the bio-tolerability of the skin contact zone and is slightly rough.

Warming of affected skin sections H being important for the treatment process depends (as presented on principle in see FIG. 6) on:
power P losses of the temperature-dependant resistor PTC (again in dependence of PTC temperature −θ_{PTC}),
thermal resistance R_{TH1} of the heat-conductive material M,
thermal resistance R_{TH2} of body K,
thermal resistance R_{TH3} of contact to skin (contact skin surface HK),
thermal capacity of the affected skin area,
losses P_{V}.

Application of governing properties of PTC makes possible to very cheaply realize working temperature within preset temperature range without any auxiliary transducers and governors. Simultaneously the required skin temperature is gained in suitable manner meaning the efficient area within the framework of a short period of 20 to 30 seconds (energy saving) ensuring that temperature higher than 65° C. (danger of burning) will not be reached in static state. This is due to matching of individual components one to the other with maximized heat stream and affected area and minimized losses on the one hand and on the other hand the final temperature is limited through controlled losses, especially by:
increase of thermal resistance toward the inefficient side and/or
reduction of thermal resistance toward the skin contact surface HK by way of reduction of the thickness of heat conductive material M and/or heat conductive body K and/or
optimization of contact with skin through surface geometry of body K/contact surface HK

Within the invention framework, the skin contact surface HK of the body K may be rounded also (mainly in connection with the moistened bite or sting affected area); the skin contact surface HK may partially have not heat conductive coating (as example, round-shaped); the body K may be produced through molding of resistor PTC as a plastic component and ensuring simultaneously extremely efficient transfer of heat. Due to the few components to be mounted, the housing/device may be produced quite automatically.

The invention is not confined to the above presented and described examples but is covering all modifications of similar application in the sense thereof. Besides, the invention has not confined by now to 1 or 10 combinations of parameters as defined in the patent requirement but may be defined with any other random combination of specific characteristics of all common announced single properties. This means that in principle any single property of the patent requirement 1 or 10 may be skipped or replaced respectively with at least another one single property specified in the announcement.

1. Hand-held device for thermal treatment of areas affected by insect sting or bite, comprising:
a heat-conductive body (K), that may come in contact with skin (H) of user and rests in a housing (G) of said device,
a temperature-dependant resistor with positive curve (PTC), that may be disposed in or on said heat-conductive body (K) and is thermally coupled with the latter,
a button component (T) actuated manually by the user and
a source of voltage (B) mounted in the housing,
such that according to said time said button component (T) is pressed down and according to said heat transfer resistors (R_{TH1}, R_{TH2}, R_{TH3}) between said temperature-dependant resistor (PTC), said heat-conductive body (K) and said skin (H) said heat-conductive body (K) and said skin contact area (HK) is heated up in a temperature range between 50° C. and 65° C. by self warming of said resistor with positive curve (PTC).

2. Hand-held device according to claim 1, wherein said body (K) made as a hollow body and said temperature dependant resistor (PTC) made in the form of disc-like washer with two terminals (A1, A2), an wherein the disc-like washer is positioned at least by injection of heat conductive material (M) into the hollow space of said heat conducting body (K).

3. Hand-held device according to claim 1, wherein said temperature dependant resistor (PTC) fixed through complete molding of one part of said body (K) by said body (K) itself.

4. Hand-held device according to claim 1, wherein said skin contact area (HK) of said body (K) is formed by a convex or concave surface.

5. Hand-held device according to claim 1, wherein said body (K) made of metal or ceramics has a gold-coated skin contact surface (HK) which has a slightly roughness.

6. Hand-held device according to claim 1, wherein said body (K) is formed at the cover with a continuous, inwards or outwards directed, profile (P) whereto another profile of two plastic half-shells of said housing (G) which is complementary is rested.

7. Hand-held device according to claim 2, wherein said body (K) made as a hollow cylinder which is open on one side and wherein said disc washer (PTC) is disposed axially with said hollow cylinder (K).

8. Hand-held device according to claim 6, wherein said first half-shell of housing (G) has a partition wall between battery (B) and said incorporated components of said device and wherein said second half-shell is divided and wherein said one section incorporating structural components of the device is connected by a locking component to the first half-shell and wherein said second section is movably rested in said first half-shell which adhering close to the shape of said first section.

9. Hand-held device according to claim 1, wherein said device is provided with LED or lamps (D) for optical indication of actuated button.

10. Method for thermal treatment of areas affected by insect sting or bite by a hand-held device with a temperature-dependant resistor with positive curve (PTC), a thermally coupled heat-conducting body (K), a voltage source (B) and a button component (T) actuated manually by the user, whereas a user:
places said hand-held device onto the affected area and
then actuates said button component (T) for thermal treatment of affected area as long as said self-heating temperature-dependant resistor (PTC) gains individually felt temperature range between 50° C. and 65° C. at said skin contact area (HK) of said heat-conductive body (K).
